(19) United States
(12) Patent Application Publication
(10) Pub. No.: US 2024/0122549 A1
(43) Pub. Date: Apr. 18, 2024

(54) LOCALIZING PHYSIOLOGICAL SIGNALS

(71) Applicant: COMPUMEDICS LIMITED, Abbotsford, Victoria (AU)

(72) Inventor: Michael WAGNER, Hamburg (DE)

(21) Appl. No.: 18/277,566

(22) PCT Filed: Feb. 25, 2021
(86) PCT No.: PCT/IB2021/051564

§ 371 Date: Aug. 16, 2023

(51) Int. Cl. A61B 5/00; A61B 5/245; A61B 5/372

(52) U.S. Cl. CPC A61B 5/7253; A61B 5/245; A61B 5/372; A61B 5/742

(57) ABSTRACT

The invention provides a method and apparatus for acquisition and analysis of data that displays a linear relationship or can be transformed into a linearized relationship, such as electrophysiological signal data from sensors such as those suitable for EEG, MEG, ECG and the like. The method, which can be implemented in computer software, includes computing a cortical current flow vector field or a distribution of activity-indicating values for cortical locations according to an existing method of choice, determining, which currents are not inward-flowing, and defining a diagonal weighting matrix whose entries representing locations where currents are not inward-flowing are smaller compared to its other entries and re-calculating the cortical current flow vector according to the method of choice but incorporating the diagonal weighting matrix, or modifying the distribution of activity-indicating values, such that values representing locations where currents are not inward-flowing indicate smaller activity than before the modification. The outputs of the method can be stored in computer files for display on suitable monitors.

### FIELD OF THE INVENTION

The present invention relates to an apparatus for acquiring electrophysiological signals associated with physiological processes, in particular, electroencephalogram (EEG) and magnetoencephalogram (MEG) measurements, and to methods for analysis of electrical signals produced in said measurements by said apparatus.

### BACKGROUND

Brain activity can be represented by data from EEGs and MEGs, which are comprised of measurements of electrical signals from electrode sensors positioned adjacent a head (EEG) or coils positioned above the surface of a head (MEG). In the analysis of acquired EEG and MEG data from sensor outputs, brain activity can be represented as a discrete three-dimensional vector field, each vector denoting a dipolar electrical current source, hereinafter referred to as a “current source”. The result provides a representation of the underlying synaptic activity of neurons in the working brain at a point in time and over time.

EEG and MEG recordings of interictal epileptiform brain activity often contain waveform morphologies known as spikes. Using source localization techniques on the onset or peak of such spikes may reveal brain locations that are involved in epileptic networks. The waveform morphology at the spike onset or peak is created by simultaneous activity of a type of neurons called pyramidal cells from an extended patch of cortical gray matter with a size of at least 10 cm² (EEG) or 6 cm² (MEG). Because the source of EEG and MEG signals are the activities of pyramidal cell neurons, and due to the dominating orientation of that specific cell type, the orientation of brain current flow is known to be perpendicular to the local cortical gray matter surface. Because spike onset and peak represent activity stemming to an overwhelming extent from the de-polarization part of their neuronal activation cycles, the direction of brain current flow is furthermore known to be inward-pointing and oriented towards the gray matter-white matter boundary. It is state of the art to incorporate as constraints into the source localization algorithm that brain activity arises from cortical gray matter only, that the orientation of cortical current flow is perpendicular to the cortical gray matter surface, and that neighbouring cortical locations have similar activity. What is needed is improvement in using the range of characteristics of measurable physiological signals to represent physiological functions.

It is known in the art that the relationship between the aforementioned vector field and the measured signals is linear. The relationship is uniquely determined by the sensor layout adjacent (EEG) or above the head (MEG), the choice of reference (ground), the measurement noise, and the electrical conducting properties of the head, known as a “forward model”. For any time point, this linear relationship can be written as A x+n=b, where A (the lead field matrix) represents the forward model and the choice of reference, n represents the measurement noise, b represents the measured data, and x represents the vector of strengths of electrical current sources, “the currents”, comprising one to three entries per discretization point. The aforementioned vector field is comprised of the unit vectors used to calculate A, each multiplied by the corresponding scalar entry of x. For convenience, in this document, symbols representing matrices are written in bold uppercase, symbols representing vectors are written in bold lowercase, the N^{th} entry of a vector, x, is identified by x_{N}, and the (M,N)^{th} entry of a matrix, A, is identified by A_{M,N}.

For a particular distribution of current sources, the sensor layout, the reference, the forward model, and, assuming no electrical noise, the measured physiological electric signal data can be predicted uniquely. This is known as the “forward problem”.

However, there is the problem that, for any chosen set of measured physiological electrical signal data, the sensor layout, the reference, and the forward model, the distribution of current sources cannot be computed uniquely due to any of the following reasons:
the number of sensors is limited; or
the noise is unknown; or
there are typically more unknown values (currents) than there are known values (sensors); or
there exist current configurations (silent sources) that produce no measurable signal.

Such a problematic situation, which is common with electrophysiological measurements, is known in the art as an ill-posed, ill-conditioned inverse problem. However, the estimation of these currents is an important objective in EEG and MEG analysis, for example, to make the output of the measurements meaningful.

Methods known in the art for computing the currents, given the measured physiological signal data, utilize a data model comprising the noise characteristics of the data and a source model comprising assumed features of the currents. The noise characteristics of the data (data model) are typically expressed using a noise covariance matrix, Cₙ, which can be estimated from the measured signal data using said assumptions. Data and the lead field matrix can also be “pre-whitened”, yielding a noise covariance matrix of 1.

A widely made assumption in the art regarding the characteristics of the currents (source model) is that most currents are small or zero. This assumption derives, for example, from the nature of an observed brain state where one localized type of activity might be assumed to dominate, or by the nature of an experiment, where many instances of data sharing a common feature of interest are averaged and, consequently, all but the observed feature is assumed to be suppressed by the averaging process. A corresponding source model is the minimum-norm least-squares model, where the L2-norm x^{T} Cₛ⁻¹ x is assumed to be minimal, with Cₛ being the source covariances of x. If no information regarding the source covariances is available, then Cₛ=1. Regularization is used to balance the influence of the data model and the source model. Following this line of reasoning, a unique xₒₚₜ, (where xₒₚₜ is the optimal vector as defined above) can be obtained by solving a linear inverse problem by minimization of the expression,

xₒₚₜ=arg min[(Ax−b)^{T}Cₙ⁻¹(Ax−b)+λx^{T}Cₛ⁻¹x],

where λ is the regularization parameter. It is well-known in the art that an analytical solution for xₒₚₜ can be obtained. Furthermore, it is well-known in the art that an optimal value of λ can be obtained based on no additional information.

It is well-known in the art that a representation of the middle layer of the individual cortical gray matter sheet (“gray matter surface”), which is where the pyramidal cell neurons reside, can be obtained from magnetic resonance imaging (MRI) data. Because the orientation of the generating neurons is locally perpendicular to the gray matter surface, it is also well-known in the art that if cortical currents shall be estimated, and the previously mentioned discretization points sample the cortical gray matter surface densely enough to account for the variability of orientations within the gray matter, the vector of electrical current sources, x, may comprise just one entry per discretization point. If the lead field matrix A is created based on unit currents that are consistently either inward- or outward-pointing, the sign of x_{N} may serve as an indicator for whether the current at location N is flowing inward (de-polarization) or outward (re-polarization). In this context, “inward” means “towards the white matter” while “outward” means “towards the pial surface”.

It is known in the art that methods exist which do not calculate a vector xₒₚₜ representing currents, but rather a vector sₒₚₜ using a metric indicating cortical locations that are likely involved in creating the events-of-interest. One example of these is the sLORETA method.

In this document, the words, “including” or “comprising”, are used interchangeably with the same meaning, which is to be not limited to any stated feature or list of features.

### SUMMARY OF THE INVENTION

The inward-pointing direction of cortical current flow has not been used as a constraint yet in analysing MEG and EEG measurements. Such a directional constraint is the subject of the invention. Using the inward-pointing direction of cortical current flow as constraint in a source localization algorithm is not an obvious extension of the state of the art, because most types of brain activity typically subjected to source localization do not stem to an overwhelming extent from de-polarization-type neuronal activity and can therefore not be characterized by inward-pointing direction of cortical current flow, with epileptic spikes an example of a notable and clinically relevant exception.

It is an object of the invention to provide a method of transforming data comprising EEG and/or MEG signal measurements to represent brain activity from de-polarizing neurons only (inward current flow). It is a further object to provide an algorithm that can be implemented in computer software to analyse electrophysiological signal data to provide a result comprising of a representation of physiological activity for interpretation and analysis. It is a further object to provide an apparatus to acquire physiological signal measurements with a linear relationship as described herein and transform the signal measurements into representations of physiological activity.

The invention provides a method for analysis of electrophysiological signal data to enable physiological interpretation of measured signals. According to the invention, physiological signal data, b, is measured and a lead field matrix, A, is computed. Furthermore, either a discrete cortical source current vector, xₒₚₜ, or a discrete metric sₒₚₜ indicating likelihood of cortical current flow is computed.

In one aspect, the invention provides a method for transforming electrical signal data from sensors using a microprocessor, including the steps of collecting and storing electrical signal data into a computer file; pre-processing the data; marking one or more time points of interest; applying an averaging step; calculating or obtaining cortical locations and corresponding neuronal orientations; calculating location weights and/or cortical currents; determining which currents are not inward-flowing; modifying weights accordingly; calculating currents according to weights; calculating a distribution of activity-indicating values for cortical locations; calculating, extracting, or estimating the direction of cortical current flow; determining which currents are not inward-flowing; modifying the distribution of values accordingly; and storing the resulting data in at least one computer file. Preferably the method includes the steps of applying a data imaging technique to the stored resulting for transforming the data into a form suitable for visual representation of the data and displaying the transformed data for visual inspection.

In another aspect, the invention provides an apparatus for collecting, transforming and displaying electrical signal data, comprising: sensors for collecting electrical signals; means for storage of electrical signal data; and at least one microprocessor having a computer program implementing pre-processing the data; marking or having the user mark one or more time points of interest; applying an averaging step; calculating or obtaining cortical locations and corresponding neuronal orientations; calculating location weights and/or cortical currents; determining which currents are not inward-flowing; modifying weights accordingly; calculating currents according to weights; calculating a distribution of activity-indicating values for cortical locations; calculating, extracting, or estimating the direction of cortical current flow; determining which currents are not inward-flowing; modifying the distribution of values accordingly. Preferably the apparatus includes means for storing transformed data. Preferably the apparatus includes means for displaying the transformed data.

### BRIEF DESCRIPTION OF THE FIGURES

FIG. 1 shows a flowchart of the method of the invention.

FIG. 2 shows an example of EEG signals with electrical impulses recorded on 25 channels in FIG. 2a and a computer-generated voltage topography plot in FIG. 2b. The outcome of Step 8 or Claim 1a and 2a is shown here.

FIG. 3 shows an example of an analysis of EEG data using the method of the invention. The outcome of Step 12 or claim 1b is shown here.

FIG. 4 shows a further example of an analysis of EEG data using the method of the invention. The outcome of Step 15 or claim 1e is shown here.

FIG. 5 shows a further example of an analysis of EEG data using the method of the invention. The outcome of Step 18 or claim 2c is shown here.

FIG. 6 shows a further example of an analysis of EEG data using the method of the invention. The outcome of Step 20 or claim 2e is shown here.

### DETAILED DESCRIPTION OF THE DRAWINGS AND BEST METHOD OF PERFORMANCE

The method is most conveniently applied to signals of EEG and MEG measurements to provide a result that shows a representation of brain activity. It will be understood that the invention is most advantageously applied to the collection and analysis of EEG and MEG data, but that the method is not limited to the analysis of EEG and MEG data, the invention having more general application such as in the application to electrocorticogram (ECoG) measurements of brain activity, intracranial (iEEG) measurements of brain activity, electrocardiogram (ECG) measurements and magnetocardiogram (MCG) measurements of heart activity, for example. The invention provides a method for analysis of data, including electrophysiological data, which displays the linear relationship described herein, or can be linearized (using, e.g., Newton's method) to do so. The invention is useful in all cases where the sign of the values in x or s is known to be zero or positive only, or zero or negative only.

According to the invention, the method can either be used to augment an existing method that calculates cortical currents, or an existing method that calculates a distribution of values that provide a metric indicating cortical locations that are likely involved in creating the events-of-interest, and in addition calculates or allows to extract or to estimate, per cortical source, the direction of current flow. In the following, both options are described.

When used to augment an existing method that calculates cortical currents, if the method allows to incorporate a weighting matrix or other mechanism that indirectly modulates the strength of the calculated cortical currents, for the purpose of the invention, this mechanism is used to assign weights to cortical sources depending on their previously calculated direction of current flow to the desired effect that calculated cortical sources without inward-pointing directions become less active. If the method is implemented so that these weights are determined iteratively based on several repetitions of a weighted inverse calculation per the definition of the specific algorithm, the additional weighting performed for the purpose of the invention can be incorporated into the existing algorithm, for example after each iteration, or in a final step following the last iteration of the existing method. If the method is not implemented as an iterative weighting scheme, after the existing method has run, the same or a similar method is repeated but now incorporating a weighting performed for the purpose of the invention, based on the cortical currents obtained in the first run.

A transformation technique known as “Source Weighting” utilizes the equality,

Cₛ=W⁻²Cₚ,

where Cₚ is the source covariance matrix of x. Cₚ encodes external prior knowledge about the source distribution. If no such information is available, Cₚ=1. The diagonal weighting matrix W is determined by the Source Weighting method itself. Given A, b, and Cₚ, different values of x are obtained depending on W. In order to determine W, the values of x calculated by the existing method are used, so that W_{N}=f(x_{N}) where the weighting function f is designed so that its values never become negative but are smaller if the value x_{N} indicates that currents are not inward-flowing, as compared to the case where the value x_{N} indicates that currents are inward-flowing. For example,
f(x)=1, if x<0; else
f(x)=0,
   assuming that inward-flowing currents at location N are identified by negative values of x_{N}. The cortical source current vector xₒₚₜ is then re-calculated using the weighting matrix W. In the actual calculations, it is typically not required to actually invert W, from which follows that negative values W_{N} are unproblematic. Should W need to be explicitly inverted as per the implementation of the existing method of choice, 1/0 shall be a large, positive number.

The method of the invention conveniently implements the herebefore described techniques into computer software for transforming electrical signal data into representations in ways not previously known to be useful.

The use of a weighting matrix is known in the art. However, weighting matrices are used in the art in order to achieve a desired amount of focality in the source distribution or to effectively minimize norms other than the L₂-norm of x. According to the invention, the weighting matrix is used to suppress non-inward-flowing currents, providing the surprising utility found in the result. The method of the invention when used with electrophysiological signal measurements, for example, EEG or MEG measurements or other suitable measurements, has not previously been shown.

The invention includes a device having electrodes for acquiring electrophysiological signal data, a means for storing said data, a means for transforming said data, a microprocessor for making calculations in the transformation, computer software implementing the algorithm of the method, a means for storing transformed data, and a means for displaying transformed data. In one embodiment, the invention comprises an EEG apparatus and electrodes for measuring an EEG, a means for electronically storing EEG data, a means for storing computer software and executing computer software implementing the invention, a means for electronically storing transformed data and a screen for displaying transformed data. The screen may be any suitable screen capable of displaying images. This may include screens on analogue or digital monitors. It will be understood that the scope of the invention includes many embodiments that will achieve the objectives.

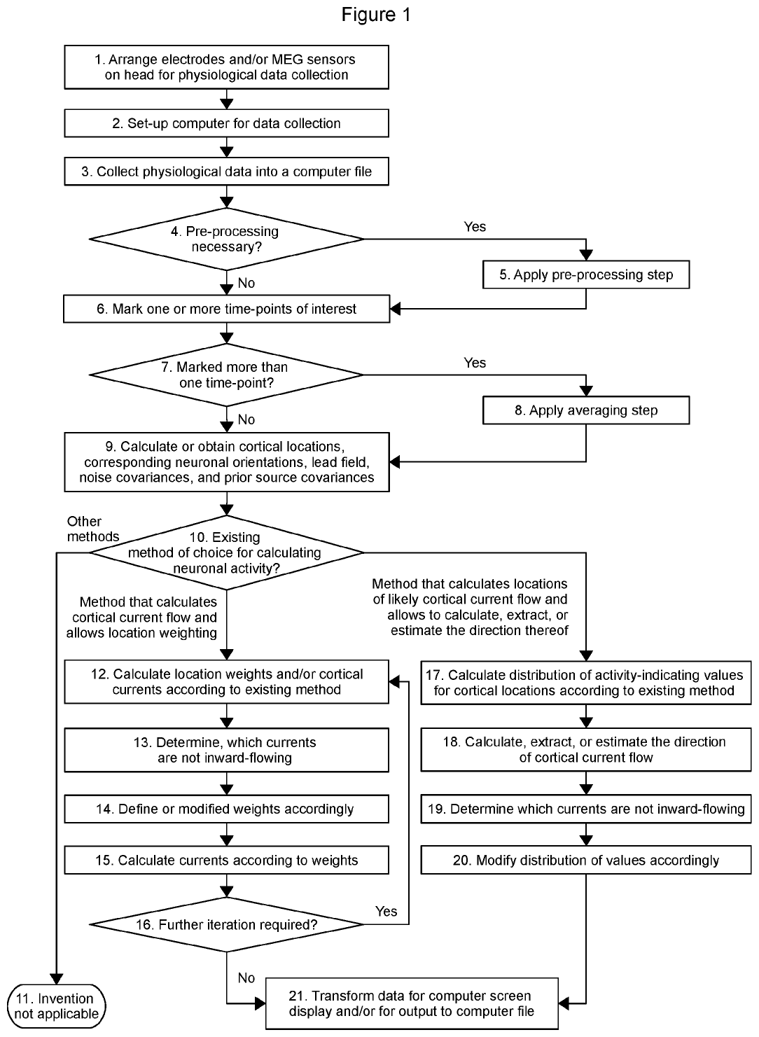

Embodiments of the method include combinations of data collection and transformation steps illustrated in the boxes in the flowchart shown in FIG. 1. Initially, sensor electrodes are arranged adjacent the head of a subject, for example, in the case of EEG and MEG 1, and a computer is set up to collect and transform outputs into computer data files 2. It will be understood that the scope of the invention includes any type of physiological signals that are suitable for use in the method as described herein. Transformed data representing electrophysiological signals is collected and/or stored for further processing 3. In processing the data, a determination is made whether or not to pre-process the data 4. The data may be pre-processed 5, or the time-point or time-points of interest may be marked without pre-processing 6. In further processing the data, a determination is made whether one or more time-points of interest have been marked 7. The data may be averaged 8, or the cortical locations and corresponding neuronal orientations may be calculated or obtained, and the noise covariances, lead field, and prior source covariances may be calculated 9 without averaging. The existing method of choice is a method that calculates cortical currents and allows location weighting 10. Subsequently, the location weights and/or cortical currents are calculated according to the existing method 12. It is determined, which currents are not inward-flowing 13. Weights W are defined or modified accordingly 14. Cortical currents are calculated, taking into account the weights W 15. A determination is made whether or not an additional iteration is required 16. The resulting data is stored in random-access memory (RAM) for further transformation by suitable data-imaging techniques for representation of the data for visual display or output to a computer file for later use 21.

More specifically, the method using Minimum Norm Least Squares (MNLS) or Focal Underdetermined System Solution (FOCUSS) or sLORETA-Weighted Accurate Minimum-Norm (SWARM) with iteration or any other weighted linear inverse solver as the existing method of choice determines the cortical source current vector, xₒₚₜ, in the following steps:
a) Collecting electrical signal data into a computer file. Optionally, applying pre-processing such as filtering.
b) Marking time-points of interest. Optionally, averaging.
c) Determining cortical locations, corresponding neuronal orientations, noise covariances Cₙ, lead field A and prior source covariances Cₚ.
d) Computing the current density vector xₒₚₜ and the final weighting matrix W_{final} based on the measured data b, noise covariances Cₙ, lead field A and prior source covariances Cₚ by executing the existing method of choice, either until successfully iterated or continuing with step e). In the case of MNLS, the number of iterations is one and W_{final}=1.
e) Computing the diagonal weighting matrix W so that its entries (one per location) are determined by a function of their corresponding values in xₒₚₜ, such that locations of non-inward-pointing current flow obtain smaller weights that locations of inward-pointing current flow, e.g.

W_{N,N}=sgn(X_{opt,N})*0.5+1.
f) Re-computing the updated current density vector xₒₚₜ based on the measured data b, noise covariances Cₙ, lead field A, diagonal weighting matrices W and W_{final} and weighted source covariances Cs=W⁻² W_{final}⁻² Cₚ by solving the related weighted linear inverse problem.
g) If the method of choice is an iterative method and in step d) the choice was made to not iterate, continue with step d) unless successfully iterated.

As an alternative to using a weighting matrix W where some W_{N,N} are set to zero, the method may in many cases also be implemented by removing the corresponding source locations, thus reducing the dimensionality of x and xₒₚₜ, and either re-calculating lead field A and prior source covariances Cₚ, or simply deleting the corresponding rows and columns.

When used to augment an existing method that calculates a distribution of values that provide a metric s indicating cortical locations that are likely involved in creating the events-of-interest, and in addition calculates, or allows to extract or to estimate, or can be supplemented by a method that calculates or allows to extract or to estimate, per cortical source, the direction of current flow, for the purpose of the invention, this mechanism is used to modify the distribution of values such that locations without inward-pointing directions of current flow indicate less likelihood of being involved in creating the events-of-interest.

According to the invention, the resulting metric sₒₚₜ is calculated based on the result of the existing method, s, and the information whether the direction of current flow at a given location N is inward-pointing or not, such that in sₒₚₜ, compared to s, locations without inward-flowing currents obtain values that indicate a lesser likelihood of being involved in creating the events-of-interest. For example,
s_{opt,N}=s_{N}, if current at location N is inward-flowing; else
s_{opt,N}=0.

The method of the invention conveniently implements the herebefore described techniques into computer software for transforming electrical signal data into representations in ways not previously thought to be useful.

According to the invention, the information about direction of cortical current flow, together with the modification of the result metric s, provides the surprising utility found in the result. The method of the invention when used with electrophysiological signal measurements, for example, EEG or MEG measurements or other suitable measurements, has not previously been shown.

The invention includes a device having electrodes for acquiring electrophysiological signal data, a means for storing said data, a means for transforming said data, a microprocessor for making calculations in the transformation, computer software implementing the algorithm of the method, a means for storing transformed data, and a means for displaying transformed data. In one embodiment, the invention comprises an EEG apparatus and electrodes for measuring an EEG, a means for electronically storing EEG data, a means for storing computer software and executing computer software implementing the invention, a means for electronically storing transformed data and a screen for displaying transformed data. The screen may be any suitable screen capable of displaying images. This may include screens on analogue or digital monitors. It will be understood that the scope of the invention includes many embodiments that will achieve the objectives.

Embodiments of the method include combinations of data collection and transformation steps illustrated in the boxes in the flowchart shown in FIG. 1. Initially, sensor electrodes are arranged adjacent the head of a subject, for example, in the case of EEG and MEG 1, and a computer is set up to collect and transform outputs into computer data files 2. It will be understood that the scope of the invention includes any type of physiological signals that are suitable for use in the method as described herein. Transformed data representing electrophysiological signals is collected and/or stored for further processing 3. In processing the data, a determination is made whether or not to pre-process the data 4. The data may be pre-processed 5, or the time-point or time-points of interest may be marked without pre-processing 6. In further processing the data, a determination is made whether one or more time-points of interest have been marked 7. The data may be averaged 8, or the cortical locations and corresponding neuronal orientations may be calculated or obtained, and the noise covariances, lead field, and prior source covariances may be calculated 9 without averaging. The existing method of choice is a method that calculates locations of likely cortical current flow and allows to calculate, extract, or estimate the direction thereof 10. Subsequently, the distribution of activity-indicating values for cortical locations is calculated according to the existing method 17. The direction of cortical current flow is calculated 18. It is determined, which currents are inward-flowing 19. The distribution of activity-indicating values is modified, based on the direction of current flow 20. The resulting data is stored in random-access memory (RAM) for further transformation by suitable data-imaging techniques for representation of the data for visual display or output to a computer file for later use 21.

More specifically, the method using sLORETA as the existing method of choice determines the metric sₒₚₜ indicating cortical locations that are likely involved in creating the events-of-interest in the following steps:
a) Collecting electrical signal data into a computer file. Optionally, applying pre-processing such as filtering.
b) Marking time-points of interest. Optionally, averaging.
c) Determining cortical locations, corresponding neuronal orientations, noise covariances Cₙ, lead field A and prior source covariances Cₚ.
d) Computing the current density vector xₒₚₜ based on the measured data b, noise covariances Cₙ, lead field A and prior source covariances Cₚ by solving the related unweighted linear inverse problem.
e) Computing the sLORETA result s based on the current density vector xₒₚₜ
f) Determining, for which locations the direction of current flow stored in xₒₚₜ is not inward-pointing.
g) Computing the metric sₒₚₜ based on the sLORETA result s by assigning values indicating a lesser likelihood of being involved in creating the events-of-interest to locations where the direction of current flow is not inward-pointing.

The method using SWARM without iteration as the existing method of choice would use the metric sₒₚₜ as opposed to the metric s before calculating the cortical currents. As an alternative and only when computing the metric sₒₚₜ based on the the sLORETA result s by assigning values indicating zero likelihood of being involved in creating the events-of-interest, the method using SWARM without iteration may also be implemented by removing the corresponding source locations, thus reducing the dimensionality of sₒₚₜ.

The method of the invention is most conveniently practised by implementing the method in a computer algorithm. In particular, there is a large amount of signal data acquired in the measurement of an EEG or MEG that must be transformed by the method of the invention to provide a meaningful result.

### Examples

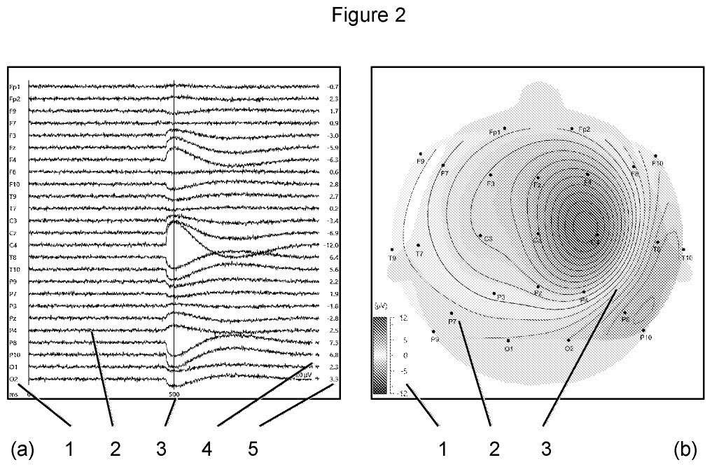

Simulated EEG data containing a point source with a source strength time-course modelling a de-polarization followed by a re-polarization phase are shown in FIG. 2. In FIG. 2a, on the left, the output 2 of 25 sensors located on the head in an EEG is shown, together with its scale 4 and each channel's amplitude in μV 5 at the time point depicted by the vertical time cursor 3 which denotes the time point used for analysis, which is the peak of the de-polarization phase. Furthermore, each sensor (channel) is labelled according to the sequence on the left-hand side 1. In FIG. 2b, on the right, a computer-generated rendering of the sensors 2 (identified by their labels) and isopotential lines of the voltages 3 for the selected time point are shown together with the scale used 1. The noise covariance matrix Cₙ is diagonal in this example, and all its non-zero entries are (0.5 μV)² which corresponds to a signal-to-noise ratio of 10. The source prior covariance matrix Cₚ is 1.

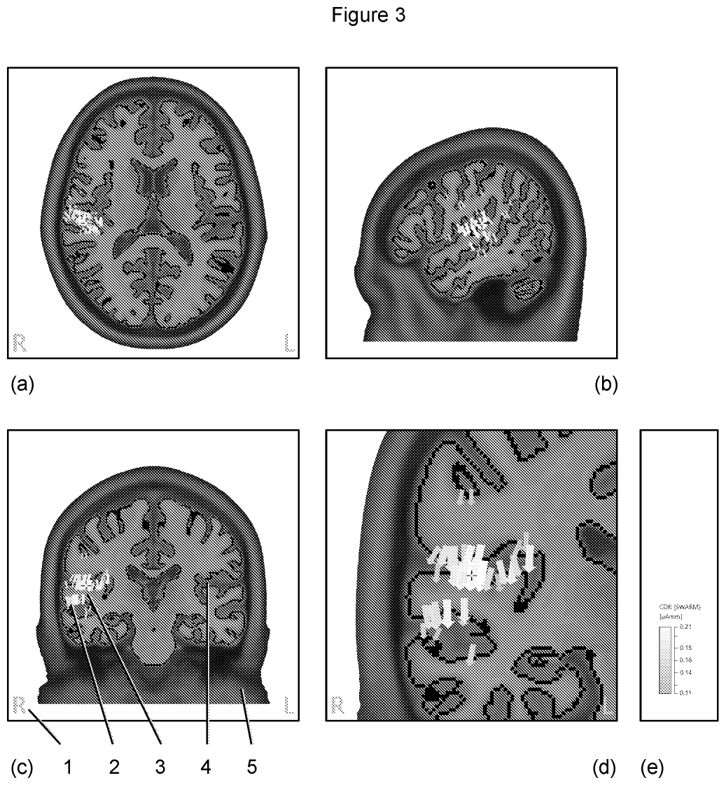

FIGS. 3 to 6 show analysis results applied to EEG signal data. In all of these figures, in parts a to c, three orthogonal cuts through the 3-D solution space show the analysis results 2. Analysis results are depicted as arrows indicating the location, orientation, and strength of the analysis result. The location represented by each arrow is the centre of the arrow, halfway between the tail and the tip. The strength represented by each arrow is indicated by the colour and also the size of the arrow. The tip of teach arrow indicates the direction of cortical current flow. Also shown are labels indicating right (“R”) 1 and left (“L”), an anatomical backdrop 5, and a surface representing a middle layer of the cortical sheet 4, on which the source locations are distributed. In the orthogonal cuts, the black crosshair 3 shows the location of the simulated point source. In part d, a magnified view of the area around the crosshair can be seen, according to part c. In part e, a scale can be seen, indicating the colours used to display the analysis result.

FIG. 3 shows the results of the existing method SWARM with iteration. The units seen at the scale are μAmm which is current dipole moment.

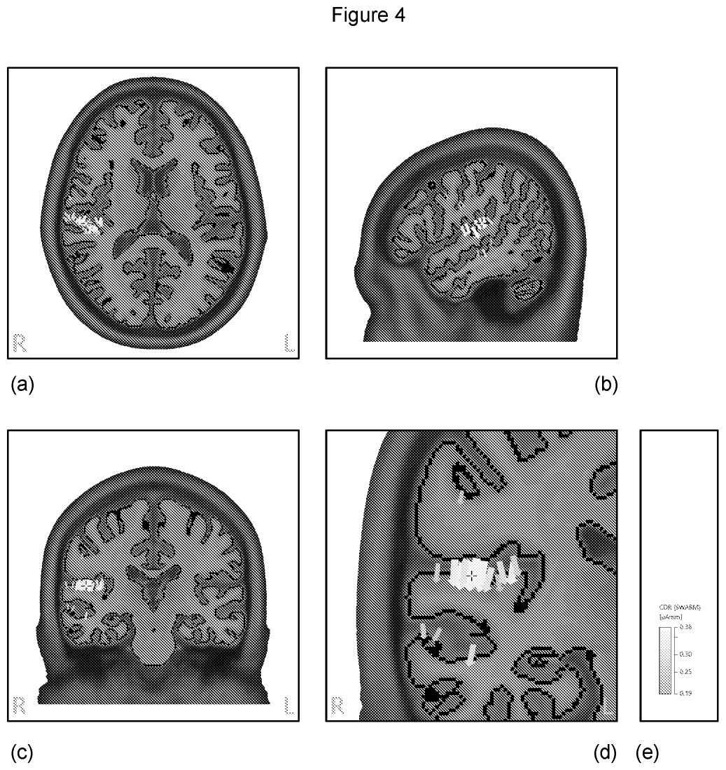

FIG. 4 shows the results of the proposed method, where the existing method is the SWARM method with iteration. The units seen at the scale are μAmm which is current dipole moment.

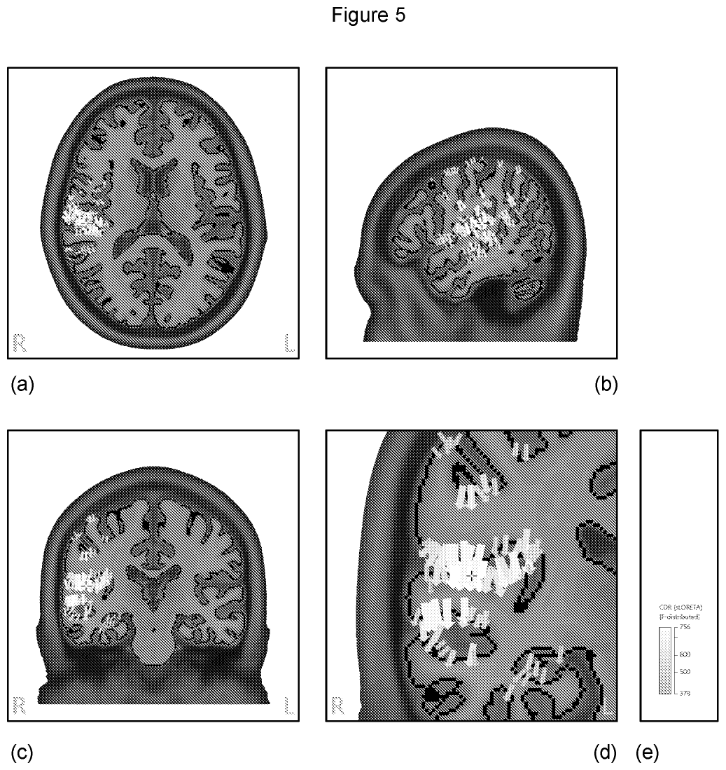

FIG. 5 shows the results of the existing method sLORETA. The units seen at the scale indicate a unitless, F-distributed statistical score.

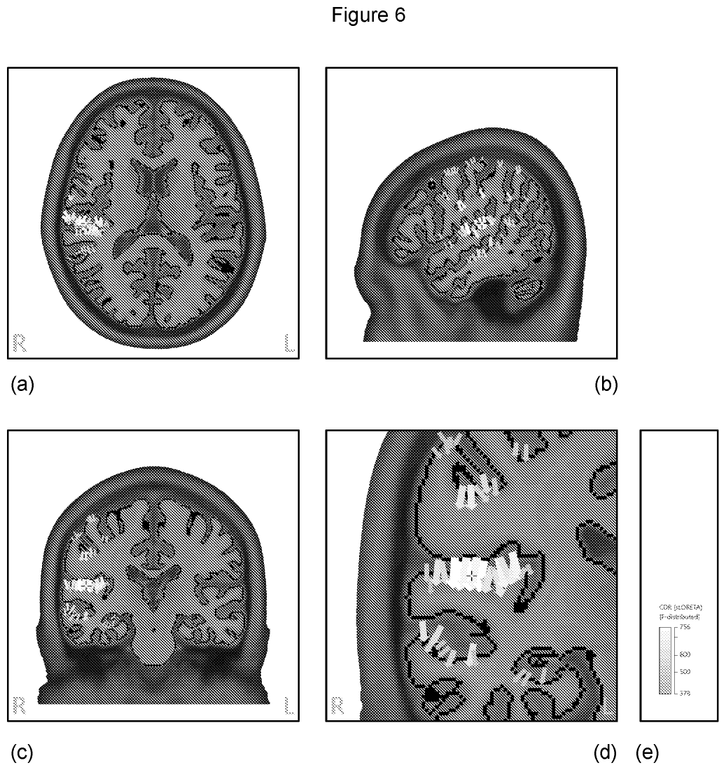

FIG. 6 shows the results of the proposed method, where the existing method is the sLORETA method. The units seen at the scale indicate a unitless, F-distributed statistical score.

### REFERENCES

Dale A. M., Sereno M. I. Improved Localization of Cortical Activity by Combining EEG and MEG with MRI Cortical Surface Reconstruction: A Linear Approach. Journal of Cognitive Neuroscience 5, 162-176, 1993.
Fuchs M., Wagner M., Köhler T., Wischmann H. A. Linear and Nonlinear Current Density Reconstructions. J Clin Neurophysiol 16, 267-295, 1999.
Gorodnitsky I. F., George J. S., Rao B. D. Neuromagnetic source imaging with FOCUSS: a recursive weighted minimum norm algorithm. Electroencephalogry and Clinical Neurophysiology 4, 231-51, 1995.
Hämäläinen M., Ilmoniemi R. Interpreting magnetic fields of the brain: minimum norm estimates. Report TKK-F-A559, Helsinki University of Technology, Espoo, 1984.
Köhler T. Lösungen des bioelektromagnetischen inversen Problems. PhD-Thesis, University of Hamburg, Hamburg, 1998.
Pascual-Marqui R. D. Review of methods for solving the EEG inverse problem. International Journal of Bioelectromagnetism 1, 75-86, 1999.
Pascual-Marqui R. D. Standardized low resolution brain electromagnetic tomography (sLORETA): technical details. Methods & Findings in Experimental & Clinical Pharmacology 24D, 5-12, 2002.
Pascual-Marqui R. D., Michel C. M., Lehmann D. Low resolution electromagnetic tomography: a new method for localizing electrical activity in the brain. International Journal of Psychophysiology 18, 49-65, 1994.
Sekihara K., Sahani M., Nagarajan S. S. Localization bias and spatial resolution of adaptive and non-adaptive spatial filters for MEG source reconstruction. NeuroImage 25, 1056-1067, 2005
Tao J X, Ray A, Hawes-Ebersole S, Ebersole J S. Intracranial EEG substrates of scalp EEG interical spikes. Epilepsia 2005; 46:669-676.
Tarantola A. Inverse Problem Theory (2nd edition). Elsevier, Amsterdam, 1994.
Wagner M. Rekonstruktion Neuronaler Ströme. Shaker Verlag, Aachen, 1998.
Wagner M, Fuchs M, Kastner J. Current Density Reconstructions and Deviation Scans Using Extended Sources. In: Biomag 2002. Eds.: H. Nowak, J. Haueisen, F. Gießler, R. Huonker, VDE Verlag, Berlin, Offenbach 2002, 804-806
Wagner M., Fuchs M., Kastner J. Evaluation of sLORETA in the presence of noise and multiple sources. Brain Topogr. 16, 277-80, 2004.
Wagner M, Fuchs M, Kastner J. (2007) SWARM: sLORETA-weighted accurate minimum-norm inverse solutions. (Eds) Cheyne D, Ross B, Stroink G, Weinberg H. New Frontiers in Biomagnetism. Amsterdam, Elsevier. pp. 185-188

1. A method for transforming electrical signal data from sensors using a microprocessor, including the steps of:
a) collecting and storing electrical signal data into a computer file; b) computing a cortical current vector according to an existing method of choice;
c) determining, which currents are not inward-flowing;
d) computing a diagonal weighting matrix whose entries representing locations where currents are not inward-flowing are smaller compared to its other entries;
e) computing the current vector according to the existing method of choice but incorporating the diagonal weighting matrix determined in the previous step; and
f) storing the resulting data in a least one computer file.

2. A method for transforming electrical signal data from sensors using a microprocessor, including the steps of:
a) collecting and storing electrical signal data into a computer file; b) calculating a distribution of activity-indicating values for cortical locations according to an existing method of choice;
c) calculating, extracting, or estimating the direction of cortical current flow;
d) determining, which currents are not inward-flowing;
e) modifying the distribution of activity-indicating values, such that values representing locations where currents are not inward-flowing indicate smaller activity than before the modification; and
f) storing the resulting data in a least one computer file.

3. The method of claim 1, further including the step of applying a data imaging technique to the stored resulting for transforming the data into a form suitable for visual representation of the data.

4. The method of claim 3, further including the step of displaying the transformed data for visual inspection.

5. Apparatus for collecting, transforming and displaying electrical signal data, comprising: sensors for collecting electrical signals; means for storage of electrical signal data; and at least one microprocessor having a computer program implementing the existing method of choice, an algorithm to determine the direction of cortical current flow, and a diagonal weighting matrix algorithm or an algorithm for modifying the distribution of activity-indicating values, for transforming stored electrical signal data.

6. Apparatus according to claim 5 further comprising means for storing transformed data.

7. Apparatus according to claim 5, further comprising means for displaying the transformed data.

8. The method of claim 2, further including the step of applying a data imaging technique to the stored resulting for transforming the data into a form suitable for visual representation of the data.

9. Apparatus according to claim 6 further comprising means for displaying the transformed data.
